SEQUENCE LISTING (6 sequences)

SEQ ID NO: 1 - RNA, 85 nt, Artificial Sequence
  Synthetic
cgcuggcgacgggacauuauuacuuuugguacgcgcugugacacuucaaacucguaccgugaguaauaaugcgccguccacggca

SEQ ID NO: 2 - RNA, 84 nt, Artificial Sequence
  Synthetic
cuccccauggcccugucucccaacccuuguaccagugcugggcucagacccugguacaggccugggggacagggaccuggggac

SEQ ID NO: 3 - RNA, 89 nt, Artificial Sequence
  Synthetic
gucagcagugccuuagcagcacguaaauauuggcguuaagauucuaaaauuaucuccaguauuaacugugcugcugaaguaagguugac

SEQ ID NO: 4 - RNA, 110 nt, Artificial Sequence
  Synthetic
ccuggccuccugcagugccacgcuccguguauuugacaagcugaguuggacacuccaugugguagagugucaguuugucaaauaccccaagugcggcacaugcuuaccag

SEQ ID NO: 5 - RNA, 82 nt, Artificial Sequence
  Synthetic
gcuucgcuccccuccgccuucucuucccgguucuucccggagucgggaaaagcuggguugagagggcgaaaaaggaugaggu

SEQ ID NO: 6 - RNA, 72 nt, Artificial Sequence
  Synthetic
cuugggaauggcaaggaaaccguuaccauuacugaguuuaguaaugguaaugguucucuugcuauacccaga

(19) United States
(12) Patent Application Publication
(10) Pub. No.: US 2015/0125864 A1
(43) Pub. Date: May 7, 2015

(54) METHODS OF STABILIZING A VESICLE IN A SAMPLE

(71) Applicant: Samsung Electronics Co., Ltd., Suwon-si (KR)

(72) Inventors: Hyun Ju KANG, Hwaseongi-si (KR); Ga-hee Kim, Yongin-si (KR); Ye-ryoung Yong, Seoul (KR); Kyung-hee Park, Seoul (KR); Jong-myeon Park, Incheon (KR)

(21) Appl. No.: 14/324,950

(22) Filed: Jul. 7, 2014

(30) Foreign Application Priority Data
Nov. 6, 2013 (KR) 10-2013-0134363

(51) Int. Cl. G01N 1/40; C12Q 1/68

(52) U.S. Cl. CPC G01N 1/405; C12Q 1/6806
USPC 435/6.12; 436/177

(57) ABSTRACT

Provided is a method of stabilizing vesicles in a sample by combining a sample comprising a vesicle with a chelating agent and a composition used in the stabilizing the vesicle in the sample.

### CROSS-REFERENCE TO RELATED APPLICATIONS

This application claims the benefit of Korean Patent Application No. 10-2013-0134363, filed on Nov. 6, 2013, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 1,803 bytes ASCII (Text) file named “716153_ST25.TXT,” created Jul. 7, 2014.

### BACKGROUND OF THE INVENTION

1. Field

The present disclosure relates to methods of stabilizing a vesicle in a sample and compositions used for stabilizing the vesicle.

2. Description of the Related Art

Microvesicles in a living organism are small vesicles with a membranous structure present in various kinds of cells or secreted from cells. Types of microvesicles secreted extracellularly include (i) exosomes, membranous vesicles of phagocytic origin, which have a diameter from about 30 nm to about 100 nm, (ii) ectosomes (shedding microvesicle (SMV)), membranous vesicles with a diameter from about 50 nm to about 1,000 nm directly released from plasma membrane, and (iii) apoptotic blebs with a diameter from about 50 nm to about 5,000 nm released from dying cells.

Exosomes have been observed via electron microscopy that do not directly detach from a plasma membrane but rather originate from a multivesicular body (MVB), which is a special region in a cell. From the MVB, the exosome is released and then secreted. That is, when there is fusion between the MVB and the plasma membrane, the vesicles are released to the external environment outside the cells. Research indicates that the exosome is released from various and diverse types of cells when they are in a normal state or a pathological state. A molecular mechanism for how the generation of the exosome occurs has not been identified, but various types of immune cells, including red blood cells, B-lymphocytes, T-lymphocytes, dendritic cells, platelets, and phagocytic cells, and tumor cells, have been known to produce and secrete exosomes while they are alive. A microvesicle, for example an exosome, may comprise microRNA (miRNA), which is useful as a biomarker for molecular diagnosis such as early cancer.

Any bias that may occur during the pre-analytical process of preparing a sample may lead to an error in analysis. For example, when a sample is contaminated by vesicles secreted by activated platelets in the blood or vesicles are damaged during sample preparation, the measured amount of vesicles may not correctly represent the real amount of vesicles present in a pathological state. In particular, in a sample from a subject in a pathological state it may be difficult to separate the disease-specific vesicles due to contamination by the platelets-derived vesicles. This could thereby distort the diagnostic results.

Accordingly, it is essential to increase the yield of vesicles obtained in a sample and maintain the stability of the obtained vesicles for the studies, diagnoses, or monitoring using the vesicles.

### SUMMARY OF THE INVENTION

Provided is a method of stabilizing a vesicle comprising stabilizing a vesicle by combining the vesicle (e.g., a biological sample comprising the vesicle) with a chelating agent. Also provided is a composition useful for stabilizing a vesicle, the composition comprising the vesicle (e.g., a biological sample comprising the vesicle) and a chelating agent.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1A is a graph illustrating the relative fluorescence intensity of a microvesicle from separated plasma according to the concentration of EDTA added in the obtained blood;

FIG. 1B is a graph illustrating the relative fluorescence intensity of microvesicles from plasma according to the concentration of EDTA added in the obtained blood of three normal persons;

FIG. 1C is a graph illustrating the relative amount of integrin-β1 present in microvesicles obtained after immunoprecipitation of the blood plasma of three normal persons by using an anti-CD9 antibody or an anti-CD83 antibody (□: CD9, ▪: CD83);

FIG. 2A is a graph illustrating the relative amount of integrin-β1 present in microvesicles obtained from the plasma according to its storage temperature (ANOVA p-value=0) before plasma separation;

FIG. 2B is a graph illustrating the relative amount of integrin-β1 present in microvesicles obtained from the plasma according to the temperature and duration of its storage before plasma separation (ANOVA p-value=0);

FIGS. 3A-3F are graphs illustrating the Cp values of quantitative RT-PCR of microRNA present in microvesicles obtained from the plasma by separation at 4° C. within 4 hours after collecting the blood by targeting hsa-miR-126, hsa-miR-150, hsa-miR-16, hsa-miR-223, hsa-miR-320a, and hsa-miR-451 (: blood plasma 1, ▪: blood plasma 2, ♦: blood plasma 3); and

FIG. 4A is a graph illustrating the relative amount (fluorescence intensity) of microvesicles obtained by filtration after repeatedly freezing and thawing (freeze-thaw cycles);

and FIG. 4B is a graph illustrating the relative amount (fluorescence intensity) of microvesicles obtained by immunoprecipitation after repeated freeze-thaw cycles.

### DETAILED DESCRIPTION OF THE INVENTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

In an embodiment of the present disclosure, there is provided a method of stabilizing a vesicle comprising stabilizing a vesicle by combining the vesicle with a chelating agent.

A vesicle refers to a membrane structure encompassed with a lipid bilayer. For example, the vesicle may be a liposome or a microvesicle. As used in this disclosure, the term “microvesicle” is interchangable with the terms “circulating microvesicle” and “microparticle.” The microvesicle is present in a cell or is secreted from a cell. The microvesicle secreted outside a cell may include an exosome, an ectosome, an apoptotic bleb, or any combination thereof. An exosome may be a membranous vesicle of phagocytic origin with a diameter from about 30 nm to about 100 nm. An ectosome may be a large membranous vesicle being directly released from a plasma membrane with a diameter from about 50 nm to about 1,000 nm. An apoptotic bleb may be a vesicle released from dying cells with a diameter from about 50 nm to about 5,000 nm. The microvesicle in a living organism may include a microRNA (miRNA) or a messenger RNA (mRNA). A surface protein on the microvescicle may be a disease-specific marker.

The vesicle may be in a biological sample. The sample may be a body fluid. For example, the body fluid may be urine, mucus, saliva, tears, blood, blood plasma, blood serum, sputum, spinal fluid, pleural fluid, nipple aspirate, lymph, respiratory tract fluid, serous fluid, urogenital fluid, breast milk, lymph secretion, semen, cerebrospinal fluid, body fluid in organs, ascites, fluid from cystic tumor, amniotic fluid, or any combination thereof. A sample may be from an individual (i.e., subject) which may be a mammal including a human. The sample may be pretreated or not pretreated. The pretreatment may be to remove intact cells, dead cells or cell debris separated from an individual subject, for example, centrifugation, dialysis, or any combination thereof.

A chelating agent refers to a substance which binds to a metal ion thereby forming a chelate compound. The chelate compound refers to a compound which has at least two coordination atoms in one molecule or ion, wherein each of the coordination atoms has a ring structure coordinated as if surrounding the metal atom or ions. The chelating agent may be, for example, ethylene diamine tetraacetic acid (EDTA), diethylene triamine pentaacetic acid (DTPA), 1,2-diaminocyclohexane tetraacetic acid (DCTA), ethylene glycol tetraacetic acid (EGTA), nitrilotriacetic acid (NTA), ethylenediamine-N,N′-bis(2-hydroxyphenylacetic acid) (EDDHA), citric acid, or any combination thereof.

The chelating agent may be added at a concentration from about 0.01 mg/mL to about 10 mg/mL relative to the sample. For example, the concentration may be from about 0.1 mg/mL to about 9.5 mg/mL, from about 0.2 mg/mL to about mg/mL, from about 0.3 mg/mL to about 8.5 mg/mL, from about 0.4 mg/mL to about 8 mg/mL, from about 0.5 mg/mL to about 7.5 mg/mL, from about 0.6 mg/mL to about 7 mg/mL, from about 0.7 mg/mL to about 6.5 mg/mL, from about 0.8 mg/mL to about 6 mg/mL, from about 0.9 mg/mL to about 5.5 mg/mL, from about 1 mg/mL to about 5 mg/mL, from about 1.1 mg/mL to about 4.5 mg/mL, from about 1.2 mg/mL to about 4.5 mg/mL, from about 1.3 mg/mL to about 4.5 mg/mL, from about 1.4 mg/mL to about 4.5 mg/mL, from about 1.5 mg/mL to about 4.5 mg/mL, from about 1.6 mg/mL to about 4.5 mg/mL, from about 1.7 mg/mL to about 4.5 mg/mL, from about 1.8 mg/mL to about 4.5 mg/mL, from about 1.8 mg/mL to about 4.4 mg/mL, from about 1.8 mg/mL to about 4.3 mg/mL, from about 1.8 mg/mL to about 4.2 mg/mL, from about 1.8 mg/mL to about 4.1 mg/mL, from about 1.8 mg/mL to about 4 mg/mL, from about 1.8 mg/mL to about 3.9 mg/mL, from about 1.8 mg/mL to about 3.8 mg/mL, from about 1.8 mg/mL to about 3.7 mg/mL, from about 1.8 mg/mL to about 3.6 mg/mL, from about 1.8 mg/mL to about 3.5 mg/mL, from about 1.8 mg/mL to about 3.4 mg/mL, from about 1.8 mg/mL to about 3.3 mg/mL, from about 1.8 mg/mL to about 3.2 mg/mL, from about 1.8 mg/mL to about 3.1 mg/mL, from about 1.8 mg/mL to about 3 mg/mL, from about 1.8 mg/mL to about 2.9 mg/mL, from about 1.8 mg/mL to about 2.8 mg/mL, from about 1.8 mg/mL to about 2.7 mg/mL, from about 1.8 mg/mL to about 2.6 mg/mL, from about 1.8 mg/mL to about 2.5 mg/mL, from about 1.8 mg/mL to about 2.4 mg/mL, or from about 1.8 mg/mL to about 2.3 mg/mL. For example, the concentration may be about 2.25 mg/mL or lower.

The term “stabilization”, as used herein, refers to maintaining a constant condition of an object or a material without changing its condition (e.g., ruptured or intact) as effectively as possible. Stabilizing a vesicle in a sample may be to maintain the vesicle in substantially the same condition as when it was obtained. For example, stabilizing a vesicle in a sample include maintaining the amount of a vesicle in a sample as it was obtained. Stabilizing a vesicle in a sample may also include maintaining the lipid bilayer, proteins, or nucleic acids in the vesicle at the same condition as when they were obtained, inhibiting or preventing decomposition or other changes. The proteins in a vesicle may be surface proteins in the vesicle. The nucleic acid in the vesicle may be microRNA (miRNA).

The sample mixed with the chelating agent can be stored at any suitable temperature. The method may further include storing the sample mixed with a chelating agent at from about 0° C. to about 30° C. For example, the sample may be one stored at from about 0° C. to about 25° C., from about 0° C. to about 20° C., from about 0° C. to about 15° C., from about 0° C. to about 10° C., from about 0° C. to about 9° C., from about 0° C. to about 8° C., from about 0° C. to about 7° C., from about 0° C. to about 6° C., from about 0° C. to about 5° C., or from about 0° C. to about 4° C.

The sample mixed with the chelating agent can be stored for any suitable amount of time. The method may further include storing the sample mixed with a chelating agent for up to about 48 hours (i.e., between about 0 hours (the moment of the combination) and about 48 hours). The method may be to store the sample for up to about 48 hours (i.e., between about 0 hours (the moment of the combination) and about 48 hours) from when the sample was obtained. When the sample is blood, the time of obtaining the sample may be the time when the blood was collected. For example, the sample may be one stored, from the time when it was obtained, for up to about 44 hours (between about 0 hours (the moment of the combination) and about 44 hours), up to about 40 hours (between about 0 hours and about 40 hours), up to about 36 hours (between about 0 hours and about 36 hours), up to about 32 hours (between about 0 hours and about 32 hours), up to about 28 hours (between about 0 hours and about 28 hours), up to about 24 hours (between about 0 hours and about 24 hours), up to about 22 hours (between about 0 hours and about 22 hours), up to about 20 hours (between about 0 hours and about 20 hours), up to about 18 hours (between about 0 hours and about 18 hours), up to about 16 hours (between about 0 hours and about 16 hours), up to about 14 hours (between about 0 hours and about 14 hours), up to about 12 hours (between about 0 hours and about 12 hours), up to about 10 hours (between about 0 hours and about 10 hours), up to about 8 hours (between about 0 hours and about 8 hours), up to about 6 hours (between about 0 hours and about 6 hours), up to about 4 hours (between about 0 hours and about 4 hours), up to about 2 hours (between about 0 hours and about 2 hours), or up to about 1 hour (between about 0 hours and about 1 hour).

The method desirably provides for the increased stability of a vesicle (e.g., a vesicle in a biological sample). Thus, the stability of a vesicle or biological sample comprising a vesicle prepared by the method described herein can be increased as compared to a vesicle obtained without performing the present method, thus providing for longer storage times without significant degradation of the microvesicles in the sample. In some embodiments, the vesicle or biological sample comprising a vesicle can be stored for at least 1 year at −80° C.

The method may further include performing from about 1 to about 12 freeze-thaw cycles (freezing then thawing) of the sample mixed with the chelating agent. For example, the method may comprise performing between 1 and 11 freeze-thaw cycles, between 1 and 10 freeze-thaw cycles, between 1 and 9 freeze-thaw cycles, between 1 and 8 freeze-thaw cycles, between 1 and 7 freeze-thaw cycles, between 1 and 6 freeze-thaw cycles, between 1 and 5 freeze-thaw cycles, between 1 and 4 freeze-thaw cycles, between 1 and 3 freeze-thaw cycles, or between 1 and 2 freeze-thaw cycles.

The method may further include separating the vesicle from the sample before or after combining the sample with the chelating agent. Various methods known in the related art may be used for separating vesicles. Vesicles may be separated, for example, by the removing of reaction mixtures, washing or any combination thereof. The separation of vesicles may be performed from about 0° C. to room temperature. The method may further include detecting the separated vesicles. The detection may be performed by using various known methods in the art. The vesicles may be detected by, for example, dyeing the vesicles, observing under an electron microscope, or using antibodies or ligands conjugated with a fluorescent material. The method may further include analyzing the vesicles. The analysis may be performed by using various known methods in the art. The vesicles may be analyzed by, for example, detecting vesicles, and analyzing proteins, glycoproteins, lipids, nucleic acids, or any combination thereof. For example, proteins may be analyzed by using immunoblotting, immunoprecipitation, chromatography, mass spectrograph, protein chips, or any combination thereof. For example, the nucleic acid may be a miRNA. The nucleic acid may be analyzed by nucleic acid amplification method, nucleotide sequencing, analysis of single nucleotide polymorphism (SNP), or any combination thereof.

In one embodiment of the present disclosure, there is provided a composition for stabilizing a vesicle in a sample which includes the vesicle (e.g., a biological sample comprising the vesicle) and a chelating agent. All other aspects of the composition are as described above with respect to the method of stabilizing a vesicle.

In another embodiment of the present disclosure, there is provided a method of stabilizing a vesicle in a sample and a method of using the same, thereby increasing the rate of obtaining vesicles (e.g., yield of vesicle isolation from a sample) and maintaining the condition of membranes or surface proteins in the vesicles.

### EXAMPLES

### Example 1

### Yield and Detection of Microvesicles in Blood According to EDTA Concentration Relative to Blood Collected

### 1-1. Preparation of Blood and Blood Plasma

Blood was collected from the vein of a healthy subject. EDTA was added to the blood to a final concentration of 0 mg/mL (control group), 1.8 mg/mL, 2.25 mg/mL, 3.3 mg/mL, or 4.5 mg/mL, respectively, and then the mixture was immediately centrifuged at 4° C. at a rate of 1,300×g (g-force) for 10 minutes by using a swing bucket rotor. The blood plasma obtained after the centrifugation was stored at −80° C. until use.

### 1-2. Yield of Microvesicles in Blood According to EDTA Concentration

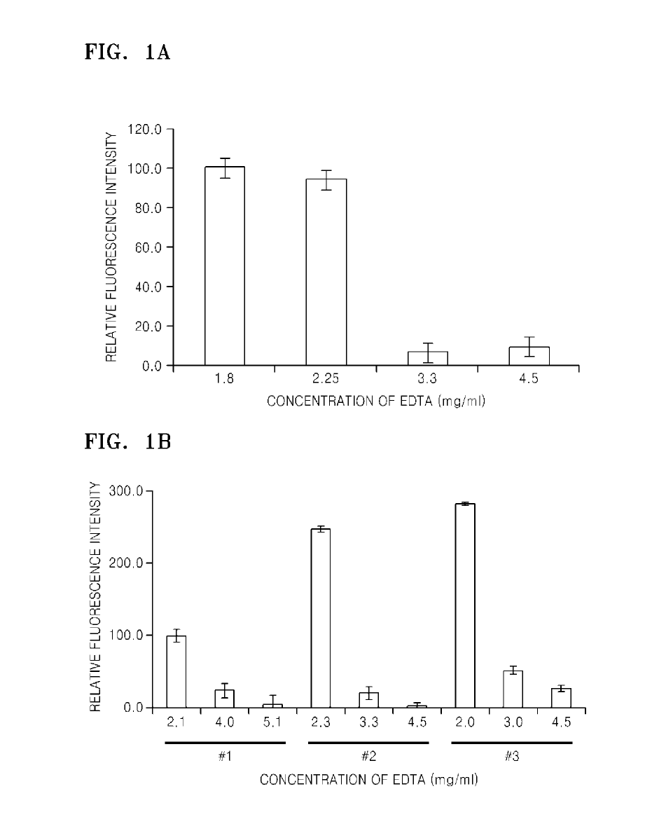

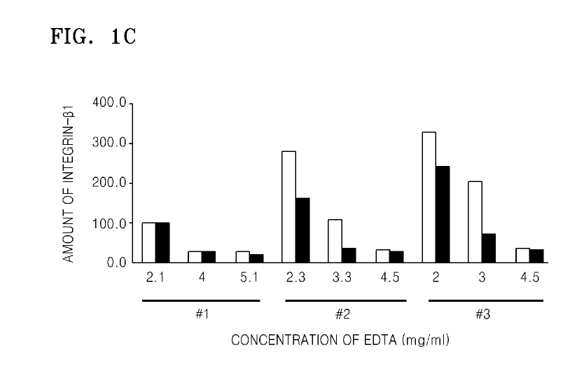

In order to confirm the presence of microvesicles in blood, a 96-well plate coated with protein G (Thermo scientific Inc.) was coated with anti-CD9 antibodies (R&D SYSTEMS, Inc.). Then, 20 μL of blood plasma obtained as described in Example 1-1 was added into each well of the 96-well plate and allowed to react for an hour. To remove floating materials not bound to the anti-CD9 antibodies, the wells were washed 3 times with PBS, 12.5 mM calcein-AM (Sigma) was added, and the plate was incubated at room temperature for 4 hours and 30 minutes to dye the microvesicles. Then, the wells were washed 3 times with PBS to remove the remaining undyed calcein-AM. To confirm the amount of dyed microvesicles, the fluorescence intensity was measured by using a fluorophotometer (Beckman, DTX800). Relative yield of microvesicles (%) was calculated by comparing the fluorescence intensity of microvesicles according to EDTA concentration added into the blood, based on the fluorescence intensity of the microvesicles in the blood plasma added 1.8 mg/mL of EDTA. The relative fluorescence intensity of the microvesicles with respect to EDTA concentration is shown in FIG. 1A, and the yield of microvesicles is also shown.

As shown in FIG. 1A, the obtained yield of the microvesicles decreased as the concentration of EDTA increased in the collected blood. More specifically, when 2.25 mg/mL of EDTA was added, the yield was about 94.1%, and when 3.3 mg/mL of EDTA was added, the yield was about 7.0%. Accordingly, it was confirmed that when the concentration of EDTA added into blood was 3.3 mg/mL or higher, the yield of the microvesicles in the blood decreased.

### 1-3. Detection Rate of Microvesicles in Blood According to EDTA Concentration

In order to calculate the detection rate of the microvesicles in blood, the microvesicles were immunoprecipitated by targeting CD8 and CD9, i.e., markers for microvesicles, and detected integrin-β1, a marker for microvesicles, and thus the immunoprecipitated microvesicles were detected.

Blood was collected from 3 normal subjects, and blood plasma was obtained as described in Example 1-1. 300 μL of the thus obtained blood plasma was mixed with either anti-CD83 antibodies (BD Pharmingen) or anti-CD9 antibodies (R&D Systems, Inc.), incubated at room temperature for 4 hours, and immunoprecipitated. As described in Example 1-2, the relative fluorescence intensity of microvesicles of the immunoprecipitated microvesicles with respect to EDTA concentration and the yield of the microvesicles calculated are shown in FIG. 1B. Immunoblotting was performed on the same sample by using anti-integrin-β1 antibodies (Abcam), and the band intensity of integrin-β1 was quantitated and the results are shown in FIG. 1C (□: CD9, ▪: CD83).

As shown in FIGS. 1B and 1C, as the concentration of EDTA added into the collected blood increased, the amount of the microvesicles immunoprecipitated with anti-CD83 antibodies or anti-CD9 antibodies decreased. Accordingly, it was confirmed that the detection rate of microvesicles obtained from blood decreased as the concentration of EDTA added into blood increased.

### Example 2

### Confirmation of Stability of Microvesicles in Blood According to Storage Temperature and Time after Blood Collection

### 2-1. Preparation of Blood Plasma

As described in Example 1-1, the blood collected and final concentration of 2.25 mg/mL of EDTA were incubated. The blood to which EDTA was added was incubated at 4° C. or room temperature for between 0 hours and 24 hours and centrifuged. Then, blood plasma was obtained therefrom.

### 2-2. Confirmation of the Amount of Microvesicles Obtained

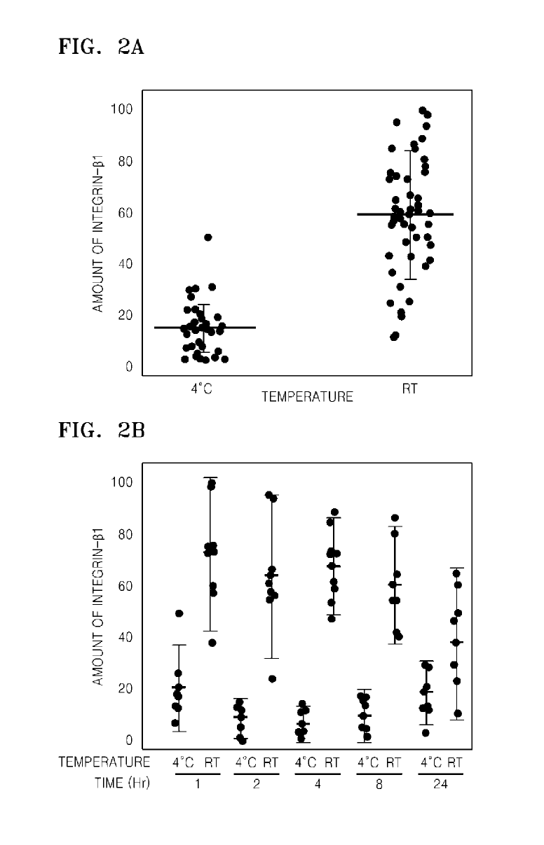

300 μL of blood plasma separated as described in Example 2-1 and anti-CD9 antibodies (R&D Systems, Inc.) were mixed, incubated at room temperature for 4 hours, and the microvesicles in blood plasma were immunoprecipitated. The immunoprecipitated microvesicles were immunoblotted by using anti-integrin-β1 antibodies (Abcam), and the band intensity of integrin-β1 was quantitated. The amount of integrin-β1 in microvesicles according to the storage temperature after blood collection is shown in FIG. 2A, and the amount of integrin-β1 in microvesicles according to the storage temperature and time is shown in FIG. 2B (ANOVA p-value=0). The amount of integrin-β1 represents the amount of microvesicles.

As shown in FIGS. 2A and 2B, no significance was observed regarding the amount of microvesicles separated from the blood and stored at 4° C. from about 4 hours to about 24 hours. However, the amount of microvesicles, which were stored at room temperature for at least 1 hour after blood collection, was significantly higher.

### 2-3. Confirmation of the Change in the Amount of miRNA in Microvesicles Obtained

As described in Example 2-2, blood was collected from 3 normal subjects, and then blood plasma was separated from the blood within 4 hours after collection at 4° C. microRNA (miRNA) was separated from the microvesicles obtained therefrom using a microRNA extraction kit (QIAGEN).

A quantitative reverse transcription-polymerase chain reaction (qRT-PCR) was performed on the separated miRNA by using a miRNA-specific primer (Exiqon, Inc). The quantitative RT-PCR was performed by targeting hsa-miR-126 (miRBase Accession No.: MI0000471), hsa-miR-150 (miRBase Accession No.: MI0000479), hsa-miR-16 (miRBase Accession No.: MI0000070), hsa-miR-223 (miRBase Accession No.: MI0000300), hsa-miR-320a (miRBase Accession No.: MI0000542), and hsa-miR-451 (miRBase Accession No.: MI0001729).

hsa-miR-126:
(SEQ ID NO: 1)
5′-CGCUGGCGACGGGACAUUAUUACUUUUGGUACGCGCUGUGACACU
UCAAACUCGUACCGUGAGUAAUAAUGCGCCGUCCACGGCA-3′
hsa-miR-150:
(SEQ ID NO: 2)
5′-CUCCCCAUGGCCCUGUCUCCCAACCCUUGUACCAGUGCUGGGCUC
AGACCCUGGUACAGGCCUGGGGGACAGGGACCUGGGGAC-3′
hsa-miR-16:
(SEQ ID NO: 3)
5′-GUCAGCAGUGCCUUAGCAGCACGUAAAUAUUGGCGUUAAGAUUCU
AAAAUUAUCUCCAGUAUUAACUGUGCUGCUGAAGUAAGGUUGAC-3′
hsa-miR-223:
(SEQ ID NO: 4)
5′-CCUGGCCUCCUGCAGUGCCACGCUCCGUGUAUUUGACAAGCUGAG
UUGGACACUCCAUGUGGUAGAGUGUCAGUUUGUCAAAUACCCCAAGUG
CGGCACAUGCUUACCAG-3′
hsa-miR-320a:
(SEQ ID NO: 5)
5′-GCUUCGCUCCCCUCCGCCUUCUCUUCCCGGUUCUUCCCGGAGUCG
GGAAAAGCUGGGUUGAGAGGGCGAAAAAGGAUGAGGU-3′
hsa-miR-451:
(SEQ ID NO: 6)
5′-CUUGGGAAUGGCAAGGAAACCGUUACCAUUACUGAGUUUAGUAAU
GGUAAUGGUUCUCUUGCUAUACCCAGA-3′

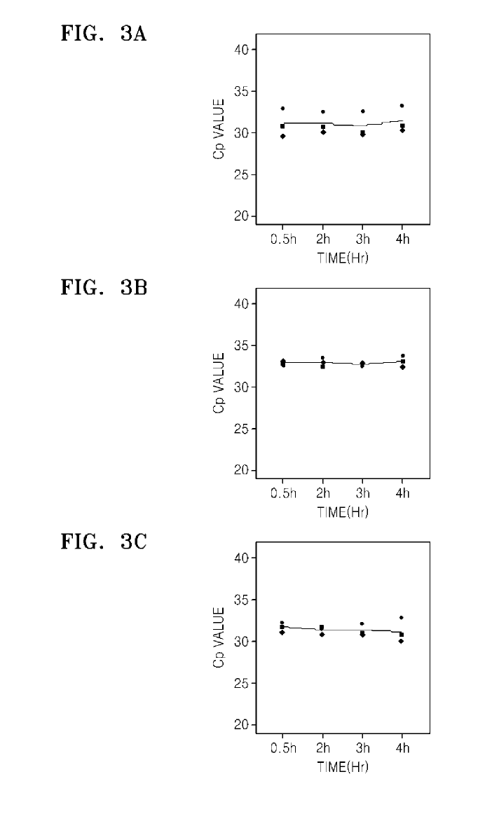

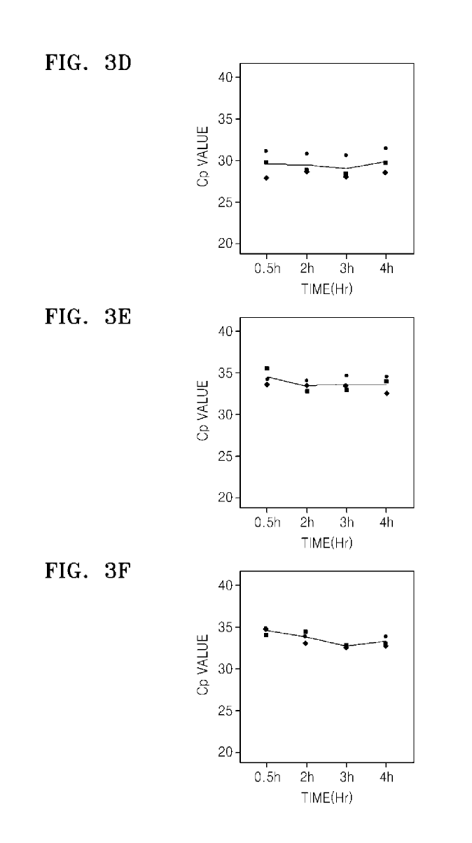

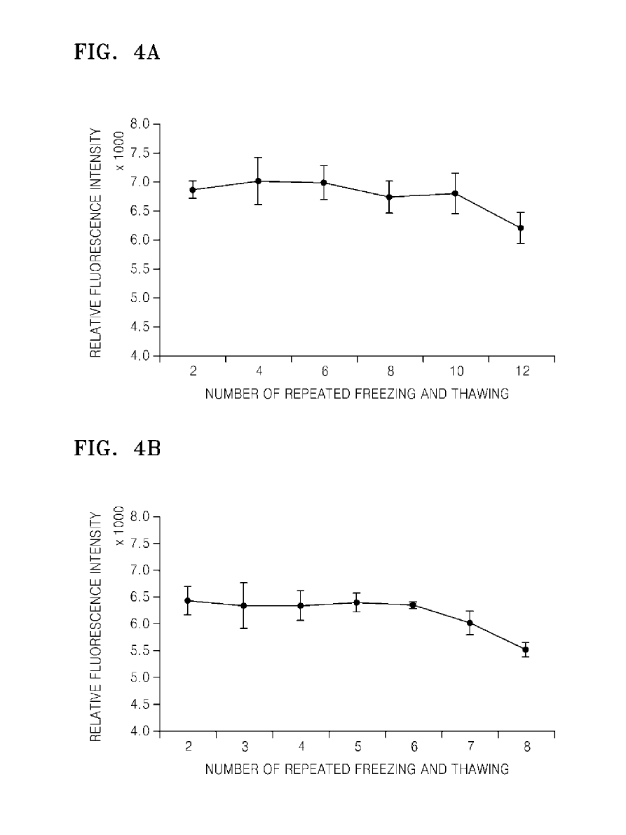

The Cp values obtained as a result of the quantitative RT-PCR are shown in FIGS. 3A-3F (FIG. 3A: hsa-miR-126(ANOVA p-value=9.75E-01), FIG. 3B: hsa-miR-150(ANOVA p-value=8.34E-01), FIG. 3C: hsa-miR-16(ANOVA p-value=9.29E-01), FIG. 3D: hsa-miR-223(ANOVA p-value=9.16E-01), FIG. 3E: hsa-miR-320a(ANOVA p-value=5.97E-01), FIG. 3F: hsa-miR-451(ANOVA p-value=1.78E-02)).

As shown in FIGS. 3A-3F, no significant change was observed in the amount of miRNA in microvesicles separated from the blood stored at 4° C. for 4 hours (: blood plasma 1, ▪: blood plasma 2, ♦: blood plasma 3).

### Example 3

### Confirmation of Stability of Microvesicles in Blood According to Repeated Freezing and Thawing

### 3-1. Preparation of Blood Plasma

To blood collected from 10 normal subjects was respectively added EDTA to a final concentration of 2.25 mg/mL, and stored at 4° C. for 4 hours. Then, the resulting mixture was centrifuged as described in Example 1-1 to obtain blood plasma, and the thus obtained blood plasma was stored at −80° C.

### 3-2. Stability of Microvesicles in Blood According to Repeated Freezing and Thawing

The blood plasma of the 10 normal subjects prepared in Example 3-1 was mixed in an equal amount. 2 mL of blood plasma and 12.5 mM calcein-AM (Sigma) were mixed, and then the vesicles (in the blood plasma) were dyed by incubating them at room temperature for 4 hours and 30 minutes. The reactants were filtrated via a 100 KDa filter (Sartorius biotechnology) to remove the remaining calcein-AM. The reactants, including the microvesicles, were aliquoted into 12 tubes in the amount of 150 μL, respectively, and repeatedly frozen (storing at −80° C. for at least 4 hours) and thawed (storing at room temperature for at least 1 hour) 1 to 12 times.

The reactants, which went through 2 to 12 freeze-thaw cycles, were filtrated via a 100 KDa filter (Sartorius biotechnology) to obtain intact microvesicles. The amount of the thus obtained microvesicles was measured via a fluorophotometer (Beckman, DTX800), and the result is shown in FIG. 4A.

As shown in FIG. 4A, no significant change was observed in the amount of microvesicles when the reactants underwent a freeze-thaw cycle 1 to 10 times.

### 3-3. Stability of the Surface Proteins of Microvesicles in Blood According to Repeated Freezing and Thawing

The blood plasma of the 10 normal subjects prepared in Example 3-1 was pooled in equal amounts. The blood plasma was aliquoted into 12 tubes in the amount of 150 μL, respectively, and repeatedly frozen (storing at −80° C. for at least 4 hours) and thawed (storing at room temperature for at least 1 hour) 1 to 12 times.

20 μL of blood plasma which went through repeated freezing and thawing was added to each well of the 96-well plate coated with anti-CD9 antibodies (R&D Systems, Inc.), as described in Example 1-2, respectively, and incubated at room temperature for 1 hour. The resulting mixture was washed 3 times with PBS to remove the floating materials which were not bound to the anti-CD9 antibodies. The resulting mixture, including vesicles, was mixed with 12.5 mM calcein-AM (Sigma) and incubated at room temperature for 4 hours and 30 minutes to dye the microvesicles. After dyeing the microvesicles, the remaining undyed calcein-AM in the microvesicles was removed by washing 3 times with PBS, their fluorescence intensity was measured via a fluorophotometer (Beckman, DTX800), and the result is shown in FIG. 4B.

As shown in FIG. 4B, the capture efficiency of microvesicles due to the anti-CD9 antibodies decreased by 14.3% to 20.8% (P<0.05) when freezing and thawing were repeated 6 times or more. Accordingly, no significant change was observed in terms of membrane stability of the microvesicles when freezing and thawing were repeated 6 times or more but it was confirmed that the surface proteins of the microvesicles were significantly damaged.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of stabilizing a vesicle comprising stabilizing a vesicle by combining the vesicle with a chelating agent.

2. The method according to claim 1, wherein the vesicle is a liposome or a microvesicle.

3. The method according to claim 2, wherein the vesicle is an exosome.

4. The method according to claim 1, wherein the vesicle is in a biological sample.

5. The method according to claim 4, wherein the sample is a body fluid.

6. The method according to claim 5, wherein the body fluid is urine, mucus, saliva, tears, blood, blood plasma, blood serum, sputum, spinal fluid, pleural fluid, nipple aspirate, lymph, respiratory tract fluid, serous fluid, urogenital fluid, breast milk, lymph secretion, semen, cerebrospinal fluid, body fluid in organs, ascites, fluid from cystic tumor, amniotic fluid, or any combination thereof.

7. The method according to claim 1, wherein the chelating agent is ethylene diamine tetraacetic acid (EDTA), diethylene triamine pentaacetic acid (DTPA), 1,2-diaminocyclohexane tetraacetic acid (DCTA), ethylene glycol tetraacetic acid (EGTA), nitrilotriacetic acid (NTA), ethylenediamine-N,N′-bis(2-hydroxyphenylacetic acid) (EDDHA), citric acid, or any combination thereof.

8. The method according to claim 1, wherein the chelating agent is added at a concentration from about 0.01 mg/mL to about 10 mg/mL relative to the sample.

9. The method according to claim 8, wherein the chelating agent is added at a concentration from about 1.8 mg/mL to about 3.3 mg/mL relative to the sample.

10. The method according to claim 1, further comprising storing the vesicle combined with the chelating agent at from about 0° C. to about 30° C.

11. The method according to claim 10, wherein the vesicle combined with the chelating agent is stored at from about 0° C. to about 5° C.

12. The method according to claim 1, further comprising storing the vesicle combined with the chelating agent from about 0 hours to about 48 hours.

13. The method according to claim 12, wherein the sample mixed with the chelating agent is stored from about 0 hours to about 4 hours.

14. The method according to claim 1, further comprising freezing and thawing the sample mixed with the chelating agent and, optionally, repeatedly freezing and thawing the sample mixed with the chelating agent up to about 12 times.

15. The method according to claim 14, wherein the method comprises repeatedly freezing and thawing the sample mixed with the chelating agent up to about 6 times of freezing or thawing.

16. The method according to claim 4, further comprising separating the vesicle from the sample before combining the sample with the chelating agent or after combining the sample with the chelating agent.

17. The method according to claim 16, further comprising detecting the separated vesicles.

18. The method according to claim 16, further comprising analyzing the separated vesicles.

19. The method according to claim 16, further comprising increasing the rate of obtaining vesicles or maintaining the condition of membranes or surface proteins in the vesicles.
